(19) United States
(12) Patent Application Publication
(10) Pub. No.: US 2026/0102138 A1
(43) Pub. Date: Apr. 16, 2026

(54) ARTERY LOCATOR

(71) Applicants: Guy P. Curtis, San Diego, CA (US); Robert Russo, San Diego, CA (US)

(72) Inventors: Guy P. Curtis, San Diego, CA (US); Robert Russo, San Diego, CA (US)

(21) Appl. No.: 18/913,080

(22) Filed: Oct. 11, 2024

(51) Int. Cl. A61B 8/08; A61B 8/00; A61B 8/06

(52) U.S. Cl. CPC A61B 8/0841; A61B 8/06; A61B 8/4455

(57) ABSTRACT

A device for establishing a fluid communication pathway to a target point in a patient includes a bi-plane ultrasound probe. A transverse plane of the probe identifies a target point in the patient, and a longitudinal plane of the probe is used to monitor 2D ultrasound signals in a segment of an artery extending downstream from the target point. By moving the longitudinal plane to optimize the received 2D ultrasound signals, a pathway to the target point is thus determined for the medical use of arteriotomy needles and/or catheters.

### FIELD OF THE INVENTION

The present invention pertains generally to systems and methods for establishing an open communication pathway to a radial artery of a patient. More particularly, the present invention pertains to transducers which generate a bi-plane ultrasound probe that includes both a transverse plane which is oriented perpendicular to blood flow direction in the artery, and a longitudinal plane which is perpendicular to the transverse plane. The present invention is particularly, but not exclusively, useful for facilitating the use of a bi-plane ultrasound probe to establish a fluid communication pathway into an artery when using arteriotomy needles, vascular catheters and/or introducer sheaths.

### BACKGROUND OF THE INVENTION

Many medical procedures require the analysis of a patient's blood or the infusion of a medicament into the vasculature of the patient, e.g. a radial artery. Normally, accessing a patient's artery is a relatively routine matter. But, there are many instances when unforeseen difficulties make a collection or infusion procedure difficult to accomplish. Paramount among these difficulties is the inability to quickly and satisfactorily establish an effective fluid communication pathway into the patient for infusing a medicament, placing a vascular catheter, or collecting a blood sample.

Typically, preplanning any invasive procedure requires the initial consideration of identifying the intended target, and its exact location in the patient. In the specific case of a blood collection procedure, or a medicament infusion setup, the target will most likely be at a location within the vasculature of the patient, such as in the lumen of an artery. Similar considerations will apply where the target is an abnormal growth or an identifiable tumor or a vein. In any event, knowing the exact location of a target is always somewhat problematic.

Ultrasound systems are known to be useable for providing visual images of internal fluid-filled tissues and organs in a person's anatomy. These images may be particularly useful for diagnostic purposes.

However, in addition to an imaging function, the present invention uses a bi-plane ultrasound system for identifying different dimensional values for an intended target. These values are then used to specifically identify the location of the intended target within the vasculature of a patient. To ascertain these dimensional values for the intended target, the present invention provides a device having a guiding structure that establishes a straight-line pathway from an extracorporeal point, directly to the intended target point in the patient. The purpose of this, of course, is to establish fluid or structural access to a target tissue in a person for medical purposes.

In light of the above, it is an object of the present invention to provide a system and method for effectively and efficiently accessing internal tissues and/or organs in a patient. Another object of the present invention is to facilitate the task of establishing a direct pathway from an extracorporeal point to a target point in the anatomy of a person. Still another object of the present invention is to effectively employ a bi-plane ultrasound probe for identifying the location of a target point for use in establishing a straight-line communication pathway to anatomical tissue at the target point. Yet another object of the present invention is to provide a device and a methodology for accurately locating a tissue target-point in a patient that is simple to manufacture, is easy to use, and is commercially cost effective.

### SUMMARY OF THE INVENTION

In general, systems and methods in accordance with the present invention are provided to establish a pathway for direct fluid communication with a peripheral artery of a patient, e.g. a radial artery. In this specific example, the pathway is directed to a preselected target point in the artery. For disclosure purposes, the target point and the artery together serve as reference points for disclosing an operation of the present invention.

The present invention includes a transducer which generates bi-plane ultrasound images. This probe includes both a transverse plane and a longitudinal plane which is perpendicular to the transverse plane. For example, when the transverse plane is oriented perpendicular to an artery in a patient it will intersect a target point in the artery, and the longitudinal plane will be oriented parallel to the blood flow direction in the artery. A receiver, which is mounted on the transducer, is used to receive and monitor return signals from both the transverse and the longitudinal planes of the ultrasound probe.

Properly positioning the transducer and its ultrasound bi-planar probe as described above is important for two reasons. First, the intersection of the probe's transverse plane establishes the operation's target point and the depth “d” of the vessel. Second, the probe's longitudinal plane is positioned to detect 2D ultrasound signals from blood flow in the artery which are also an indication of the depth “d” of the artery.

Structurally, the transducer includes a needle guide which is supported on the transducer at an adjustable entry angle θ. Typical values for the entry angle θ will be in a range of approximately 30°-40°. For its operation, the transducer is positioned against the patient, and it is manually moved to place an entry point from the needle guide directly above the artery. This also generally aligns the longitudinal plane parallel with the artery and it positions the transverse plane perpendicular to the artery.

In an operation of the present invention, the strength of 2D ultrasound signal in the range of 3-10 MHz is used as an indication of the distance between the probe's longitudinal plane and the artery. Specifically, the depth “d” of an artery can also be determined when the strength of 2D ultrasound signals is optimized. Moreover, based on the angle θ of the needle guide, the target point location in the artery can be established.

Another possible use of the 2D ultrasound signals for the present invention is to determine whether the vessel is an artery or a vein. This determination is based on the velocity of blood flow in the vessel, and is done by assigning an artificial color to the signals to facilitate image interpretation. Specifically, the velocity of blood flow in arteries is higher than in veins. Importantly, this difference is distinguishable by the colors assigned to the signals.

A control unit is included with the transducer for moving the longitudinal plane in a direction parallel to the transverse plan. These movements will also vary the distance of the longitudinal plane from the artery. Thus, as the longitudinal plane is moved, the strength of 2D ultrasound signals received by the control unit will vary.

In summary, a depth “d” for the target point can be determined when the 2D ultrasound return signal is optimized. Also, depending on the angle θ of the needle guide, and the needle insertion length “l” from the entry point at the transducer to the target point in the artery can be determined mathematically by the equation d/l=sin θ. As envisioned for the present invention, with this information a fluid pathway to the target point in the artery can be established for continued use in several different medical protocols, regardless whether an arteriotomy needle or a catheter is involved. These dimensions are of particular importance for designing the construction of a system in accordance with the present invention.

### BRIEF DESCRIPTION OF THE DRAWINGS

FIG. 1 is a perspective view of a system and its components for establishing a fluid communication pathway to a target point in a radial artery;

FIG. 2 shows the geometric relationship of the transverse and longitudinal planes of the bi-plane ultrasound probe;

FIG. 3 is an operational diagram of the system's components;

FIG. 4 is a three dimensional geometric presentation of interactive variables in their relationship with each other for an operation of the present invention;

FIG. 5 is an elevation view of a transducer operationally positioned over a target point in the lumen of an artery.

### DESCRIPTION OF THE PREFERRED EMBODIMENTS

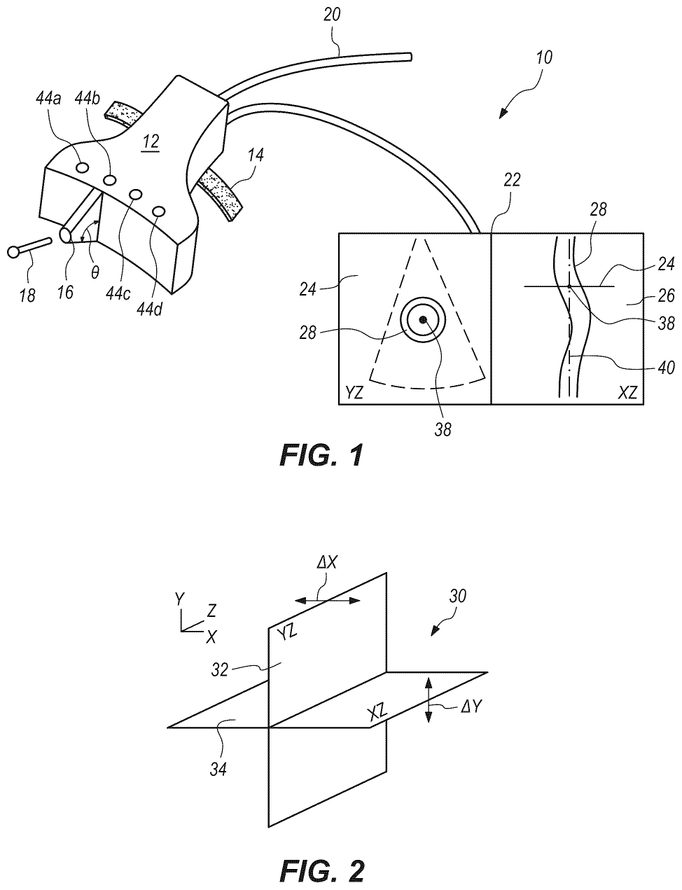

Referring initially to FIG. 1, a device for establishing a fluid communication pathway into a patient (not shown) is generally designated 10. As shown, the device 10 includes a transducer 12 which is equipped with a placement band 14 to help stabilize the device 10 on the patient. Also shown is a needle guide 16, which is mounted on the device 10 and is oriented thereon at an adjustable inclination angle θ. Typically, the inclination angle θ which will be in a range between 30°-40°. A medical device 18 is shown which can be passed through the needle guide 16. As envisioned for the present invention, the medical device 18 may be either an arteriotomy needle or a guide wire for a catheter, depending on the purpose of the device 10. Further, the needle guide 16 can be formed with a bevel notch for engagement with the medical device 18 to operationally orient and stabilize the medical device 18.

FIG. 1 also shows that the device 10 includes a power cord 20 which in used to power a display monitor 22. In detail, the display monitor 22 presents both a transverse image 24 and a longitudinal image 26 of a same target tissue in the patient. For disclosure purposes, this target tissue will hereinafter be referred to simply as an artery 28.

FIG. 2 shows a bi-plane ultrasound probe 30 for the device 10. This probe 30 is generated by the transducer 12 and it is used to produce images of tissue inside the patient. For example, the transverse image 24 and the longitudinal image 26 of an artery 28 are shown in FIG. 1. As shown in FIG. 2, the transverse plane 32 and the longitudinal plane 34 are mutually perpendicular to each other. Moreover, as intended for the present invention, the planes 32/34 can be moved respective to each other. Specifically, with reference to x-y-z Cartesian coordinates, the transverse plane 32 (yz) can be independently moved through distances ±Δx relative to the longitudinal plane 34. Similarly, the longitudinal plane 34 (xz) can be independently moved through distances ±Δy relative to the transverse plane 32.

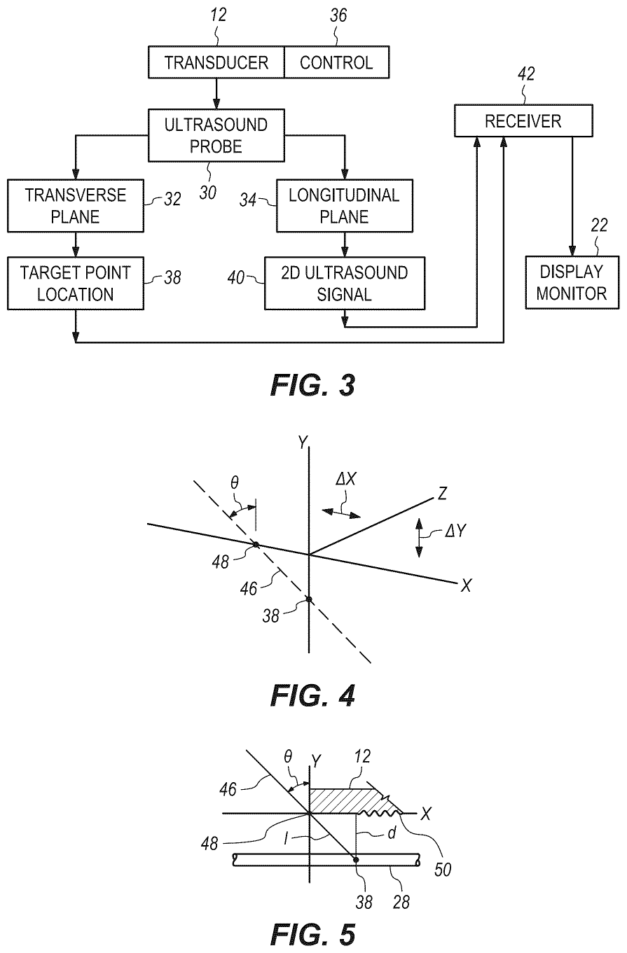

With reference to FIG. 3, essential components for a device 10 are shown with their operational characteristics interconnected. Specifically, FIG. 3 shows that the transducer 12 is interactive with a control unit 36. Note, it is the transducer 12 that generates the ultrasound probe 30.

As shown and disclosed above in FIG. 2, the ultrasound probe 30 is generated by the transducer 12 to include both the transverse plane 32 and the longitudinal plane 34. The transverse plane 32 and the longitudinal plane 34, however, interact together in the ultrasound probe 30 for different purposes. Namely, it is the transverse plane 32 that identifies the target point 38 for the medical device 18 in the transverse image 24 of the display monitor 22. On the other hand, the longitudinal plane 34 identifies the artery 28 in the longitudinal image 26. In this combination, the control unit 36 can be operated to move the transverse plane 32 and the longitudinal plane 34 relative to each other.

Operationally, as a first step, the transverse plane 32 of ultrasound probe 30 can be manually moved with the transducer 12 on the patient in directions ±Ax. This movement will thereby establish a location for the target point 38 in the transverse image 24 of the display monitor 22. Once the target point 38 has been established, the longitudinal axis 40 of the artery 28 can be displayed in the longitudinal image 26 of the display monitor 22. A receiver 42, which may be incorporated into the transducer 12, or established as a stand-alone component, is provided to receive images of both the target point 38 and the longitudinal axis 40 from the ultrasound probe 30 for presentation on the display monitor 22.

As indicated above, the location of the target point 38 can be selectively determined by manually moving the transducer 12. Locating the depth “d” of the target point 38 and of the longitudinal axis 40 of the artery 28, however, is another matter. Specifically, for this purpose, depth buttons 44a-d on the transducer 12 can be selectively activated to move the longitudinal plane 34 in directions ±Δy through a range of 2-3 mm. This movement will thereby establish the depth “d” of the artery 28 based on the intensity of 2D ultrasound signals received from blood flow in the artery 28.

FIG. 4 provides a geometric reference for identifying a fluid communication pathway 46, shown as a dashed line, which is operationally created by the device 10. As shown, the pathway 46 is oriented by the inclination angle θ of needle guide 16 on the transducer 12. The pathway 46 further extends from the needle guide 16 and through an entry point 48 on the patient, to the target point 38 in the patient. Further, as indicated on FIG. 4, the location of the fluid communication pathway 46 from the needle guide 16 and into a patient will depend on where the transverse plane 32 and the longitudinal plane 34 of probe 30 are positioned by the device 10. Specifically, movements of the probe 30 in Δx place the transverse plane 32 (yz), and movements in Δy place the longitudinal plane 34 (xz).

FIG. 5 shows a transducer 12 positioned to establish a fluid communication pathway 46 to a target point 38 in an artery 28 of a patient. For purposes of the present invention, FIG. 5 shows that the pathway 46 is essentially designed by the inclination angle θ of the needle guide 16, and the depth “d” determined by the ultrasound probe 30 of device 10. With these established values, a distance “l” along the pathway 46 from the entry point 48 to the target point 38 can be determined by the mathematical equation d/l=sin θ. To stabilize the transducer 12, while measurements are being taken, the transducer 12 can be formed with notches 50 to prevent movements of the transducer 12 on the patient.

While the particular device for establishing a fluid communication pathway to a target point in an artery of a patent as shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A device for establishing a fluid communication pathway to a target point in a peripheral artery of a patient, wherein the device comprises:
a transducer for generating a bi-plane ultrasound probe, wherein the probe includes a transverse plane and a longitudinal plane perpendicular to the transverse plane, wherein the transducer is positioned against the patient to orient the longitudinal plane parallel to a segment length of the artery, and to orient the transverse plane perpendicular to the blood flow direction in the artery;
a receiver mounted on the transducer for receiving signals from the probe that include a signal from the transverse plane for locating the target point in the artery, and a 2D ultrasound signal from the longitudinal plane indicative of blood flow in the artery;
a control unit on the transducer for moving the longitudinal plane in a direction parallel to the transverse plane until a 2D ultrasound signal from the longitudinal plane is maximized to thereby identify a depth for the target point in the patient; and
a medical device advanced to the target point to establish the fluid communication pathway into the artery.

2. The device of claim 1 wherein a plurality of depth buttons are mounted on the transducer for moving the longitudinal plane to the depth required for identifying the target point.

3. The device of claim 2 wherein depth values used for display on the monitor are adjustable in a range of 2-5 mm.

4. The device of claim 1 further comprising a display monitor for creating real-time dynamic images of an advancement of the medical device to the target point in the artery.

5. The device of claim 1 wherein the medical device is an arteriotomy needle and the medical device further comprises:
a needle support mounted on the transducer; and
a needle guide affixed to the needle support and oriented thereon at a predetermined angle for guiding the arteriotomy needle to the target point in the artery.

6. The device of claim 5 wherein the arteriotomy needle is formed with an orientation notch for a mating engagement with a bevel notch on the needle guide to align the distal end of the arteriotomy needle in the artery of the patient at a predetermined angular orientation.

7. The device of claim 5 further comprising:
a catheter; and
a guidewire, wherein the guidewire is inserted through the arteriotomy needle to position a distal end of the guidewire in the artery beyond the target point, and wherein the arteriotomy needle is then withdrawn from the guidewire to advance the catheter over the guidewire and into the artery.

8. The device of claim 1 wherein the ultrasound transducer is formed with a plurality of ridges positioned against the patient to stabilize and inhibit movement of the transducer on the patient during an operation of the device.

9. A device for establishing fluid communication with a peripheral artery of a patient at a target point in the artery, wherein the artery defines a blood flow direction in the artery, and wherein the device comprises:
a means for generating a bi-plane ultrasound probe, wherein the probe includes a transverse plane to be oriented perpendicular to the blood flow direction in the artery, with a longitudinal plane perpendicular to the transverse plane and oriented parallel to the blood flow direction in the artery;
a means for orienting the ultrasound probe relative to a segment of the artery, with the longitudinal plane parallel to the direction of blood flow in the artery;
a means for receiving a 2D ultrasound signal indicative of blood flow in the artery, wherein the 2D ultrasound signal is detected in the longitudinal plane of the ultrasound probe, and wherein the target point is detected in the transverse plane of the ultrasound probe; and
a means for moving the longitudinal plane in a direction perpendicular to the blood flow direction until the 2D ultrasound signal is optimized in the longitudinal plane to thereby identify a depth for the target point and for the artery segment.

10. The device of claim 9 wherein a plurality of depth buttons are mounted on the transducer for moving the longitudinal plane to the depth required for identifying the target point, and wherein depth values for identification are adjustable in a range of 2-5 mm.

11. The device of claim 10 which comprises:
an arteriotomy needle;
a needle support mounted on the ultrasound transducer; and
a conical needle guide affixed to the needle support and oriented thereon at a predetermined adjustable angle for guiding the arteriotomy needle to the target point in the artery.

12. The device of claim 11 wherein the arteriotomy needle is formed with an orientation notch for a mating engagement with a bevel notch on the conical needle guide to align the distal end of the arteriotomy needle in the artery of the patient at a predetermined angular orientation.

13. The device of claim 12 further comprising:
a catheter; and
a guidewire, wherein the guidewire is inserted through the arteriotomy needle to position a distal end of the guidewire in the artery beyond the target point, and wherein the arteriotomy needle is then withdrawn from the guidewire to advance the catheter over the guidewire and into the artery.

14. The device of claim 13 wherein the ultrasound transducer is formed with a plurality of ridges to inhibit movement of the transducer and stabilize the transducer on the patient during an operation of the device.

15. A method for connecting a medical device in fluid communication with a radial artery of a patient which comprises the steps of:
positioning an ultrasound transducer against the patient;
generating a bi-plane ultrasound probe with the ultrasound transducer, wherein the probe includes a transverse plane and a longitudinal plane which are mutually perpendicular to each other;
receiving a 2D ultrasound signal at the transducer for blood flow in the artery, wherein the 2D ultrasound signal is detected in the longitudinal plane of the ultrasound probe and the location of the longitudinal plane is detected as depth values in the transverse plane;
controlling movements of the longitudinal plane with the transducer until the 2D ultrasound signal is optimized in the longitudinal plane; and
using the intersection location between the longitudinal plane and the transverse plane, where the 2D ultrasound signal in the longitudinal plane, is optimized as an indication of the depth of the artery.

16. The method of claim 15 wherein the controlling step is accomplished with a plurality of depth buttons which are mounted on the transducer for moving the longitudinal plane relative to the transverse plane.

17. The method of claim 16 wherein depth values determined in claim 15 are adjustable in a range of 2-5 mm.

18. The method of claim 17 further comprising the step of creating real-time images of movements of the longitudinal plane and 2D ultrasound ty signals.

19. The method of claim 18 wherein the medical device is an arteriotomy needle.

20. The method of claim 18 wherein the medical device is a catheter.
